(19) United States
(12) Patent Application Publication
(10) Pub. No.: US 2026/0306766 A1
(43) Pub. Date: Oct. 8, 2026

(54) DEVICE FOR ADMINISTERING A DRUG

(71) Applicant: ADRACARD PTY LTD, Warrnambool, VC (AU)

(72) Inventors: Lachlan Kenneth Timms, Warrnambool (AU); Liam James Mahony, Warrnambol (AU)

(21) Appl. No.: 19/682,878

(22) Filed: May 20, 2026

Related U.S. Application Data
(63) Continuation of application No. PCT/AU2024/051372, filed on Dec. 19, 2024.

(30) Foreign Application Priority Data
Dec. 19, 2023 (AU) 2023904120

(51) Int. Cl. A61M 15/08; A61M 15/00

(52) U.S. Cl. CPC A61M 15/08; A61M 15/0035; A61M 15/0063; A61M 2202/064

(57) ABSTRACT

Embodiments relate to devices for administering drugs. The device comprises: a housing; a plurality of first chambers located in the housing; a plurality of plungers movable within the first chambers from a first position to a second position to increase a pressure of fluid contained in the first chambers; a second chamber configured to contain the drug and having an outlet; a releasable seal located between the plurality of first chambers and the second chamber, the outlet of the second chamber being in selective fluid flow communication with the plurality of first chambers via the releasable seal, wherein moving the plurality of plungers from the first position to the second position actuates release of the releasable seal to release the fluid under pressure from the plurality of first chambers into the second chamber, thereby entraining the drug in the fluid and causing the drug to be discharged from the outlet.

### CROSS-REFERENCE TO RELATED APPLICATIONS

The present application claims the benefit of priority under 35 U.S.C. §119 of Australian Provisional Patent Application No. 2023904120 filed on Dec. 19, 2023, the contents of which are incorporated herein by reference in their entirety.

### TECHNICAL FIELD

The present disclosure relates to a device for administering a drug such as a drug comprised in a powder composition.

### BACKGROUND

Many different devices are known for delivering a drug, such as orally inhaled or nasal drug products, including metered dose inhalers (MDIs), dry powder inhalers (DPIs), soft mist inhalers and nasal sprays. However, the internal mechanisms and volumetric requirements for these devices can dictate a minimum physical size and/or shape that, at the least, make these devices inconvenient to stow or carry on a person.

It is desired to address or ameliorate one or more shortcomings or disadvantages associated with existing drug delivery devices, or to at least provide a useful alternative.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

According to an aspect of the present disclosure, a device for administering a drug is provided, the device comprising: a housing; a plurality of first chambers located in the housing, each of the first chambers for containing a fluid under pressure; a second chamber configured to contain the drug and having an outlet, a means to release the fluid under pressure from the plurality of first chambers into the second chamber, thereby entraining the drug in the fluid and causing the drug to be discharged from the outlet for administration.

In some embodiments, the device may comprise means for pressurising the fluid in the plurality of first chambers. In alternative embodiments, fluid may be provided in each of the first chambers that is pressurised prior to use of the device.

According to an aspect of the present disclosure, a method of administering a drug to a person is provided, the method comprising:

pressurising a fluid contained in a plurality of first chambers; releasing the fluid under pressure from the plurality of first chambers into a second chamber containing a drug and having an outlet; and entraining the drug in the fluid in the second chamber and causing the drug to be discharged from the outlet for administration.

According to an aspect of the present disclosure, a device for administering a drug is provided, the device comprising: a housing; a plurality of first chambers located in the housing, each of the first chambers comprising a fluid; a plurality of plungers, each plunger of the plurality of plungers movable within a respective first chamber of the plurality of first chambers from a first position to a second position to increase a pressure of the fluid contained in the first chamber; a second chamber configured to contain the drug and having an outlet; at least one releasable seal located between the plurality of first chambers and the second chamber, the second chamber including the outlet in selective fluid flow communication with the plurality of first chambers via the releasable seal,

wherein moving the plurality of plungers from the first position to the second position actuates release of the releasable seal to release the fluid under pressure from the plurality of first chambers into the second chamber, thereby entraining the drug in the fluid and causing the drug to be discharged from the outlet for administration.

According to an aspect of the present disclosure, a method of administering a drug is provided, the method comprising: pressurising a fluid contained in a plurality of first chambers located in a housing using a plurality of plungers, each plunger of the plurality of plungers movable within a respective first chamber of the plurality of first chambers from a first position to a second position to pressurise the fluid contained in the first chamber; releasing at least one releasable seal located between the plurality of first chambers and a second chamber containing the drug, the second chamber including an outlet in selective fluid flow communication with the plurality of first chambers via the releasable seal, entraining the drug in the fluid in the second chamber and causing the drug to be discharged from the outlet for administration.

Embodiments may include any one or more of the features defined in the dependent claims.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments of the present disclosure will now be described, for illustrative purposes only, with reference to the accompany drawings in which:

FIG. 1 shows (a) an isometric, (b) a front, (c) a side, and (d) a cross sectional view along line A-A, of a device for administering a drug according to an example embodiment of the present disclosure, with a plurality of plungers of the device in a first position;

FIG. 2 shows (a) an isometric, (b) a front, (c) a side, and (d) a cross sectional view along line B-B, of the device of FIG. 1 with the plurality of plungers in a second position;

FIG. 3 shows an enlarged cross section M of FIG. 2(d);

FIG. 4 shows an enlarged cross section L of FIGS. 1(d) and 2(d) when the plurality of plungers are in (a) the first position and (b) the second position, respectively;

FIG. 5 shows (a) an isometric, (b) a front, (c) a side, and (d) a cross sectional view along line C-C, of a device for administering a drug according to an example embodiment of the present disclosure, with a plurality of plungers in a first position;

FIG. 6 shows (a) an isometric, (b) a front, (c) a side, and (d) a cross sectional view along line D-D, of the device of FIG. 5, with the plurality of plungers in a second position;

FIG. 7 shows (a) an isometric, (b) a front, (c) a side, and (d) a cross sectional view along line E-E, of a device for administering a drug according to an example embodiment of the present disclosure comprising an actuation prevention mechanism;

FIG. 8 shows an enlarged cross section M of FIG. 7(d);

FIG. 9 shows a partially transparent isometric view of a nozzle and manifold region of a device for administering a drug according to an example embodiment;

FIG. 10 shows (a) an isometric, (b) a side, and (d) a cross sectional view along line A-A, of a device for administering a drug according to an example embodiment of the present disclosure comprising a drug cartridge, with a plurality of plungers of the device in a first position;

FIG. 11 shows (a) a side, (b) a cross sectional view along line A-A, (c) a front, and (d) a cross sectional view along line B-B, of the device of FIG. 10, with the plurality of plungers of the device in a second position;

FIG. 12 shows (a) a close up view of the cross sectional view of FIG. 11(b), (b) an isometric view of the cross section of FIGS. 11(b), and (c), a similar view to FIG. 12(b) illustrating an alternative guide cone, according to some embodiments;

FIG. 13 shows (a) an isometric view, and (b) a cross sectional view of the device of FIG. 10 with protective caps coupled to the device;

FIG. 14 shows a device for administering a drug according to an example embodiment of the present disclosure showing (a) an exploded isometric view of protective caps in a disassembled configuration, and (b) an isometric view of the protective caps assembled with the device;

FIG. 15 shows (a) a front disassembled view, (b) a top end view, (c) a side view with deformed guide cone, and (d) a side view with expanded guide cone, of the device of FIG. 14;

FIG. 16 shows an isometric cross sectional view of the assembled device as shown in FIG. 14(b);

FIG. 17 shows (a) an isometric cross sectional view of the assembled device as shown in FIG. 14(b) with plungers in a first position, and (b) a close up isometric cross sectional view of the device of FIG. 14 without protection caps and with plungers in a second position;

FIG. 18 shows a cartridge, according to some embodiments, showing (a) a cross sectional view of the sealed cartridge, (b) an isometric view of the sealed cartridge, (c) a cross sectional view of the cartridge with seals released, (d) an isometric view of the cartridge with seals released, (e) a cross sectional view of the sealed cartridge with an alternative membrane seal, (f) an isometric cross sectional view of the sealed cartridge, (g) an isometric cross sectional view of the cartridge with seals released, and (h) an isometric cross sectional view of the cartridge with seals further open;

FIG. 19 shows a cartridge, according to some embodiments, showing (a) a cross sectional view of the sealed cartridge, (b) an isometric cross sectional view of the sealed cartridge, (c) an isometric cross sectional view of the cartridge with ball seal released and membrane seal sealed, and (d) an isometric cross sectional view of the cartridge with both seals released; and

FIG. 20 shows a cartridge, according to some embodiments, showing (a) a cross sectional view of the sealed cartridge, (b) a cross sectional view of the cartridge with seals released, (c) an isometric cross sectional view of the sealed cartridge, (d) an isometric cross sectional view of the cartridge with slide pin seal partially displaced and contacting ball seal, and (e) an isometric cross sectional view of the cartridge with slide pin seal released having also dislodged and released the ball seal.

### DETAILED DESCRIPTION

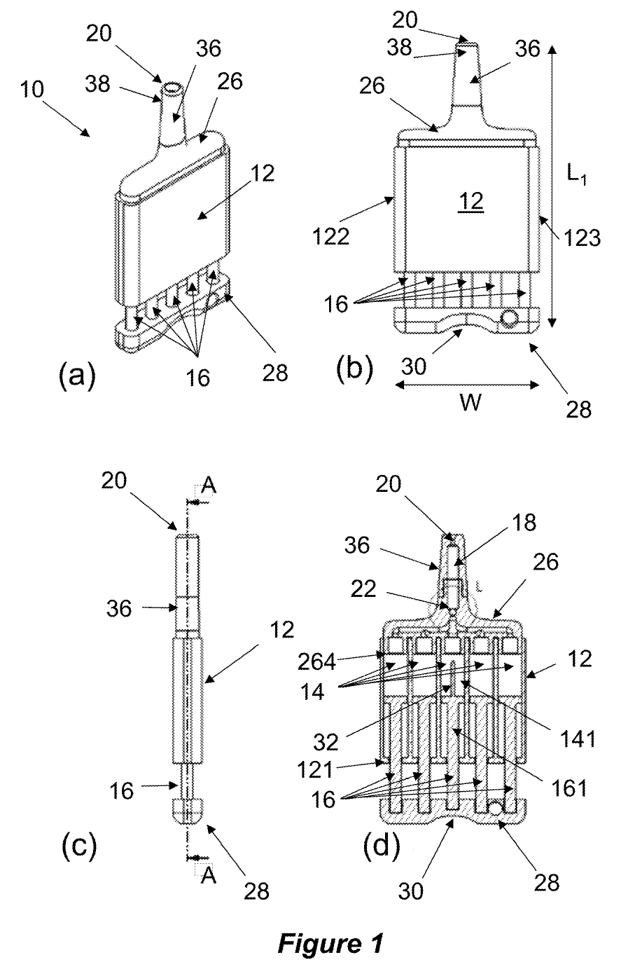

A device 10 for administering a drug according to an embodiment of the present disclosure is illustrated in FIGS. 1 to 4. The device 10 includes a housing 12, a plurality of first chambers 14 located in the housing 12 for containing a fluid under pressure in the first chambers 14. The device 10 also includes a second chamber 18 configured to contain the drug, e.g. in powdered drug form, and having an outlet 20 and a means 22 to release the fluid under pressure from the plurality of first chambers 14 into the second chamber 18, thereby entraining the drug in the fluid and causing the drug to be discharged from the outlet 20 for administration.

A drug in the present context may include one or any combination of active ingredients, mineral supplements, vitamins, excipients, formulations and drug products. The drawings show non-limiting example embodiments of a device 10 for administering a drug in the form of, or contained in a composition of, an orally or nasally delivered powder. In some embodiments the device 10 may be a dry powder inhaler (DPI). It will however be appreciated that a device 10 within the scope of the present disclosure may be used for a wide range of drugs in many forms. The drug may be used for a range of conditions in which a dose of medication may be required in emergencies and/or to prevent, mitigate or reverse a disease process. For example, the drug may be adrenaline, which may be used to treat anaphylaxis, or the drug may be a vaccine. Moreover, the drug may not necessarily be inhaled by the user. Merely as one example, the device may administer drug to the mucosa of the mid sinuses where it will be absorbed into the bloodstream without necessarily being inhaled.

It will further be appreciated that fluid in the present context refers to any substance that has no fixed shape and which yields to external pressure, such as a gas or liquid. In a preferred embodiment the fluid is a gas, such as air.

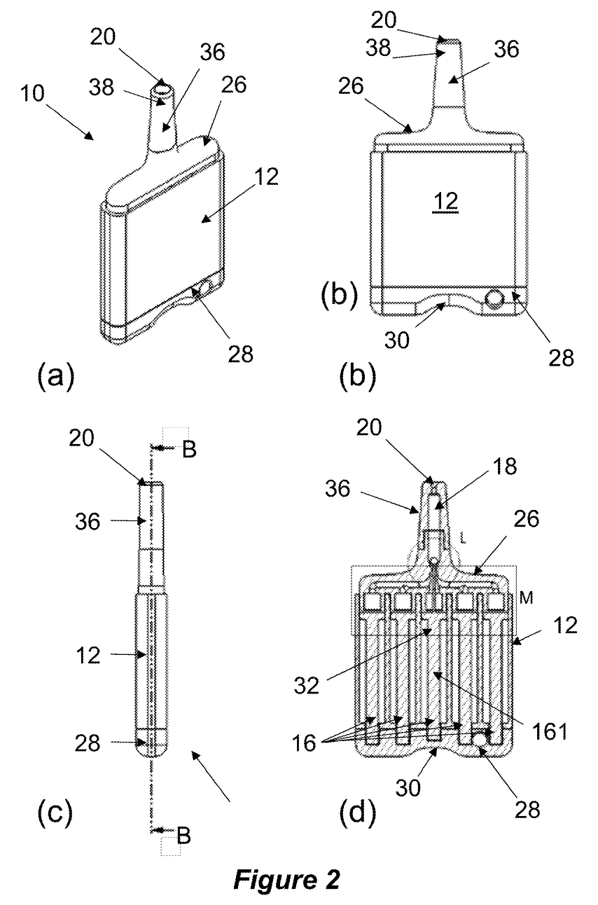

Referring to FIGS. 1 and 2, the device 10 of the present embodiment includes a non-cylindrical housing 12 having a substantially rectangular cross sectional profile when viewed from the front (or rear). Each of the first chambers 14 is substantially cylindrical and arranged in a parallel configuration in the housing along a width W direction of the housing 12. Each of the first chambers 14 comprises fluid as air.

In this embodiment, the first chambers 14 are formed by respective substantially cylindrical inner surfaces of the housing 12, e.g., inner surfaces of respective bores of the housing 12, but in alternative embodiments the first chambers may be defined by inner surfaces of respective barrels that may be located within the housing.

As a means 16 for pressuring fluid in the first chambers 14, the device 10 comprises a plurality of plungers 16, each plunger 16 of the plurality of plungers 16 being movable within a respective first chamber 14 of the plurality of first chambers 14 from a first position (as shown in FIG. 1) to a second position (as shown in FIG. 2) to increase a pressure of the fluid contained in the first chamber 14.

The device 10 includes a nozzle 36 in which at least part of the second chamber 18 is provided. As shown in FIGS. 1(d) and 2(d), the entire nozzle 36, or at least part of the nozzle 36, may be a part that is separately formed from, and fixed to, the housing 12 and/or to a manifold 26 of the device 10, although the nozzle 36 may be integrally formed in one-piece with the housing 12 and/or manifold 26 in alternative embodiments. The outlet 20 of the second chamber 18 is disposed at a discharge end 38 of the nozzle 36 in this embodiment, and adapted to be received in a mouth and/or nose of a person. In some embodiments, the nozzle 36 may be releasable from or movable relative to other parts of the device 10 (e.g. relative to the housing 12 and/or manifold 26). This may allow the introducing of, or reintroducing of, a drug into the second chamber 18 and/or allow a replacement nozzle to be fitted.

To release the fluid under pressure from the plurality of first chambers 14 into the second chamber 18 when the plurality of plungers 16 are moved from the first position to the second position, the device 10 of the present embodiment comprises a releasable seal 22. In this embodiment, as shown in greater detail in FIG. 4, the releasable seal 22 is provided by an interference fit ball. The releasable seal 22 is located between the plurality of first chambers 14 and the second chamber 18 such that the second chamber 18 and its outlet 20 are in selective fluid flow communication with the plurality of first chambers 14 via the releasable seal 22. In particular, the releasable seal 22 is located at an inlet 24 of the second chamber 18.

The manifold 26 of the device 10 is provided at a distal end of the housing 10 and fluidly connects each first chamber 14 of the plurality of first chambers 14 to the inlet 24 of the second chamber 18 via the releasable seal 22. The manifold 26 may be a part that is separately formed from, and fixed to, the housing 12 and/or to the nozzle 36 of the device 10, although the manifold 26 may be integrally formed in one-piece with the housing 12 and/or nozzle 36 in alternative embodiments.

In the present disclosure, the term “distal” or “distally” is used to describe elements of the device 10 that are located in relative terms towards the outlet 20 of the device 10 (i.e. towards the administration end of the device) and reference to “proximal” or “proximally” is used to describe elements of the device 10 that are located in relative terms towards the opposite end of the device 10 from the outlet 20.

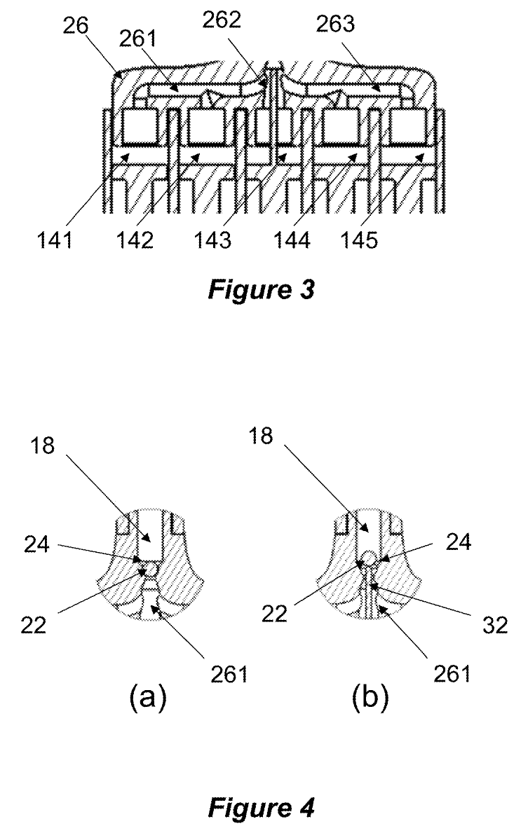

In this embodiment, with reference to FIG. 3 for example, five first chambers 14 are provided in the housing 12, including a central first chamber 143, first and second outer first chambers 141, 145 located on opposite sides of the central first chamber 143, and first and second inner first chambers 142, 144 located on opposite sides of the central first chamber 143 and between the central chamber 143 and the first and second outer first chambers 144, 145, respectively. The manifold 26 includes a central manifold channel 262 and first and second outer channels 261, 263. The central manifold channel 262 is configured to receive fluid directly from the central first chamber 143. The first outer channel 261 is configured to receive fluid from both the first outer chamber 141 and the first inner chamber 142 before channelling that fluid to the central manifold channel 262. Similarly, the second outer channel 263 is configured to receive fluid from both the second outer chamber 145 and the second inner chamber 144 before channelling that fluid to the central manifold channel 262. The central manifold channel 262 has a larger diameter than each of the first and second outer channels 261, 263 in order to accommodate the greater total amount of fluid received from both of the first and second outer channels 261, 263.

It will be recognised that the device may have an alternative number of first chambers and corresponding plungers but particularly when the device has a different odd number of first chambers and corresponding plungers (e.g. 3, 7 or more), a similar configuration of a central first chamber and further inner/outer first chambers on opposite sides of the central first chamber may be employed, in conjunction with a similarly configured arrangement of manifold channels.

The channels 261, 262, 263 of the manifold 26 may converge smoothly or seamlessly, e.g. without any sharp internal corners or edges, and may be configured to minimise any unusable volume or “dead space” in the manifold 26 that might otherwise lead to an undesirable reduction in pressure of fluid delivered from the plurality of first chambers to the second chamber 18.

The device 10 includes a handle 28 that is connected to each of the plurality of plungers 16 at each proximal end of the plungers 16 and in this regard may be considered a common handle 28. The plurality of plungers 16 are movable by the handle 28 from the first position as shown in FIG. 1 to the second position as shown in FIG. 2, and particularly through a user engaging and pressing or pushing the handle 28 in a distal direction. As shown in FIG. 2, the device 10 is configured to prevent movement of each of the plurality of plungers 16 within the respective first chamber 14 beyond the second position by means of the handle 28 being configured to abut a portion 121 of the housing 12 (e.g., a proximal end surface 121 of the housing 12) when each plunger 16 is in the second position and/or by means of a distal end of one or more of the plurality of plungers 16 being configured to abut one or more ledges 264 adjacent a distal end of the respective first chamber 14. The one or more ledges 264 may be provided by one or more proximal end surfaces of the manifold 26, for example. As a result, movement of each plunger 16 beyond the second position is prevented.

The device 10 includes an actuator 32, configured to actuate release of the releasable seal 22 when the plurality of plungers 16 are moved from the first position to the second position. In this embodiment, the actuator 32 is provided by a projection 32 extending from a distal end of a first (central) plunger 161 of the plurality of plungers 16. In this embodiment, moving the plurality of plungers 16, including the first plunger 161, from the first position to the second position causes the actuator 32 to interfere with the releasable seal 22 to release the seal 22, in particular by the projection 32 extending through the central channel 261 of the manifold 26 and pressing against and dislodging the ball of the releasable seal 22 from the interference fit as shown in greater detail in FIGS. 4(b).

Accordingly, moving the plurality of plungers 16 from the first position to the second position causes both the pressurisation of the fluid in the plurality of first chambers 14 and subsequent release of the releasable seal 22 to release the fluid once placed under pressure, from the plurality of first chambers 14, into the second chamber 18. This in turn causes entraining of the drug located in the second chamber 18 in the fluid and causes the drug to be discharged from the outlet 20 for administration.

By providing a plurality of first chambers 14, such as the above described substantially cylindrical first chambers 14, the device 10 can have a non-cylindrical housing 12 while still allowing the fluid to be effectively pressurised in each of the first chambers 14 by leveraging sealing and pressure distribution capabilities inherent to circular or cylindrical shapes. This in turn may allow a reduced physical size and shape requirement relative to the known MDIs, DPIs and nasal sprays.

In some embodiments, the device 10 may have a size that enables it to fit easily in a storage location prior to use such as in a pocket, wallet or phone case accessory. For example, the device 10 may have a credit-card-like sizing and/or appearance, at least when viewed from the front and/or rear, and including when, for example, an optional cap (not shown) is located over the nozzle 36 and/or manifold 26. The device may follow the ISO/IEC 7810 ID-1 standard that governs the size of cards like ID and credit cards, allowing for a length L₁ dimension of less than 86mm when the plurality of plungers 16 are in the first position and a width W dimension of less than 55mm. For example, the device 10 may have a length L₁ dimension of 85.60 mm and a width W dimension of 53.98 mm. Nevertheless, it is recognised that the device 10 may have e.g., a larger size than this, while still being smaller and/or different in shape than one or more known MDIs, DPIs and nasal sprays. As seen in FIG. 1(b), the length L₁ dimension extends between proximal and distal ends of the device 10 and the width direction W extends perpendicular to the proximal and distal ends of the device 10 between lateral edges 122, 123 of the device 10.

The housing 12 or parts thereof may be formed from a suitably rigid or sturdy material such as a plastic which may prevent warping or bulging of the housing 12 and generally protect the plurality of first chambers 14.

The handle 28 may be configured to comprise a rest 30, which in this embodiment is provided by a recess in a surface of the handle 28. The recess 30 is located at a centre of the handle 28 in the width direction W of the device 10, and therefore proximate the first plunger 161 of the central first chamber 141. In this manner, and when moving the plurality of plungers 16 by means of the handle 28 using a person’s finger or fingers, the person’s finger or fingers can be influenced by the rest 30 to press at a central region of the handle 28 and therefore achieve relatively symmetrical or uniform movement of the plurality of plungers 16. This may contrast with a scenario where a user might readily press, instead, an outer region of the handle 28, which may cause asymmetrical or non-uniform movement of the plurality of plungers 16 from the first position to the second position and which may lead to various issues such as jamming and inadequate pressurisation of the fluid in the first chambers 14.

The device 10 may be configured so that movement of the plurality of plungers 16 from the first position to the second position actuates release of the releasable seal 22 when the fluid is substantially at a predetermined pressure in the plurality of first chambers 14. The predetermined pressure may be selected such that the fluid flow rate through the second chamber 18 upon release of the pressurised fluid from the plurality of first chambers 14 ensures effective and consistent drug discharge from the outlet 20. Towards this purpose, the actuator 32 as a projection extending from the distal end of the first plunger 161 may be configured to extend through the central channel 262 of the manifold 26 and actuate release of the releasable seal 22 only when the first plunger has moved at least 50%, at least 60%, at least 70%, at least 80% or at least 90% of a distance between the first position and the second position. This distance may be selected to correspond to a pressure of fluid that when released from the plurality of first chambers is sufficient to ensure that substantially all of the drug contained in the second chamber 18 is entrained in the fluid and discharged from the outlet 20 for administration. In the present example embodiment, the predetermined pressure can be a pressure sufficient to ensure that the fluid flows over and through the powdered drug located in the second chamber 18 and transports substantially all of the powdered drug through the outlet 20 at a velocity suitable for oral or nasal administration.

In an embodiment, the device 10 may be configured so that a complete movement of the plurality of plungers 16 from the first position to the second position actuates release of the releasable seal 22, and accordingly that the fluid is at the predetermined pressure in the plurality of first chambers 14 substantially when the plurality of plungers 16 reach the second position. In an embodiment where the device 10 is “credit-card” sized, complete movement of the plurality of plungers 16 from the first position to the second position may, by example, involve moving each of the plurality of plungers 16 by approximately 10 mm within the respective first chamber 14. It may accordingly be necessary to ensure that the plurality of plungers 16 do not move past the second position, which can be prevented, by example, through configuration of the handle 28 such that it abuts the portion 121 of the housing 12 when each plunger 16 is in the second position as described above. This may further ensure that the projection 32 does not travel past a point where it could obstruct the outlet 20 and/or influence fluid flow over and through the powdered drug and lead to a failure in suitable administration of the drug.

From the above description, it can be understood that the device 10 may be configured to provide optimal pressurisation of the fluid in the plurality of first chambers 14, e.g. based on a volume of the first chambers 14 and a distance between the first position and the second position, and therefore allow discharge of the drug for administration at a velocity and volume that is effective and appropriate for a particular person or persons. The device 10 may be configured to achieve this in a consistent manner and while minimising user error.

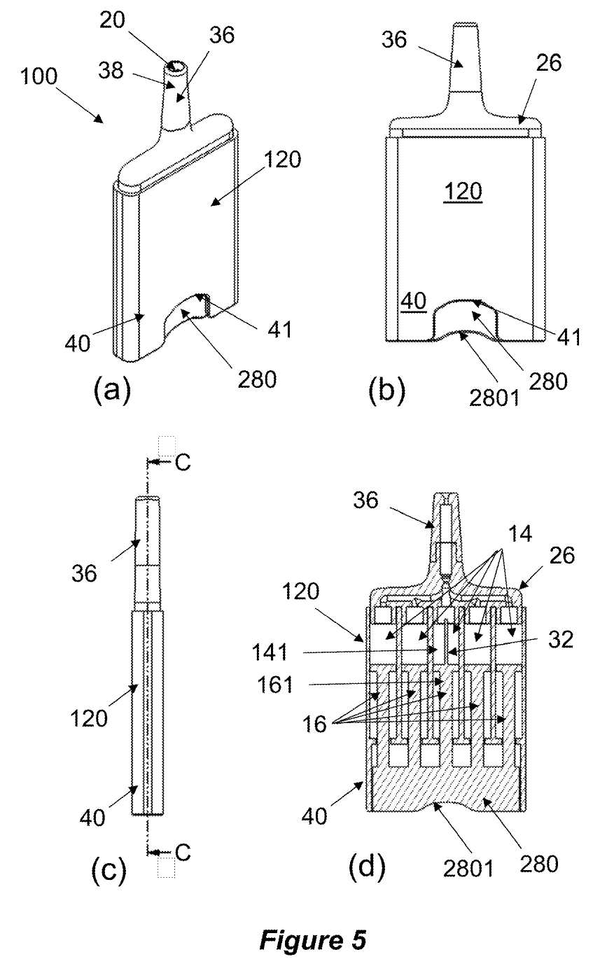

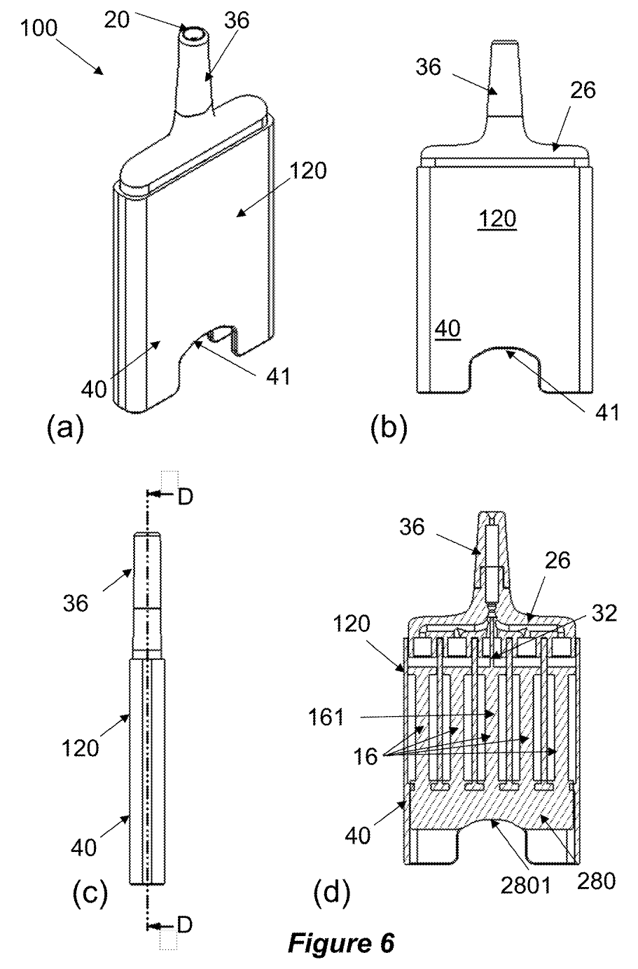

Referring now to FIGS. 5 and 6, a device 100 according to another embodiment of the present disclosure is illustrated. The device 100 is similar to the device 10 described with reference to FIGS. 1 to 4 and like reference numerals are intended to designate like features. The device 100 of this embodiment differs from the device 10 of the previous embodiments in relation to the configuration of its housing 120 and handle 280, and the relationship between the housing 120 and the handle 280.

In this embodiment, the device 100 comprises a cover portion 40 that conceals proximal ends of the plungers 16 and at least part of the handle 280 when the plurality of plungers 16 are in the first position (shown in FIG. 5). The cover portion 40 also conceals a greater part, or all, of the handle 28 when the plurality of plungers 16 are in the second position (shown in FIG. 6). The cover portion 40 is provided by a proximal part (e.g. recess) of the housing 120 in this embodiment, but it will be appreciated that this need not be the case and the receiving portion 40 may be separate from the housing 120 in alternative embodiments.

In addition to concealing components of the device 100, the cover portion 40 may guide movement of the handle 280 to move the plurality of plungers 16 substantially uniformly and symmetrically from the first position to the second position. The guiding function of the cover portion 40 may be achieved by configuring the handle 280 to slidably fit into the cover portion 40, e.g. in a relatively tight manner. In this embodiment, the cover portion 40 includes a recess 41 that receives a user’s fingers as they press the handle 280 into the cover portion 40 to move the plurality of plungers 16 from the first position to the second position. The recess 41 is provided at a centre of the cover portion 40 in the width direction W of the device 100, and therefore proximate the first plunger 161 of the central first chamber 141. In this manner, and when moving the plurality of plungers 16 by means of the handle 280 using a person’s finger or fingers, the person’s finger or fingers are required to press at a central region 2801, e.g. a recessed central region 2801, of the handle 280, leading to relatively uniform or symmetrical movement of the plurality of plungers 16.

Accordingly, the cover portion 40 may assist in guiding and therefore preventing issues such as jamming of the plungers 16. Furthermore, by the cover portion 40 concealing the proximal ends of the plurality of plungers 16, a risk of a finger or item being caught within the device mechanisms may be reduced. Still further, since the handle 280 may be substantially concealed in its entirety by the cover portion 40 when the plurality of plungers 16 are in the second position, the cover portion 40 may not only assist in providing a clear indication to a person that the device 100 has been operated and is no longer ready for use, but also limit any opportunity for a user to return the plurality of plungers 16 to the first position, e.g. by preventing grasping and moving of the handle 280 proximally.

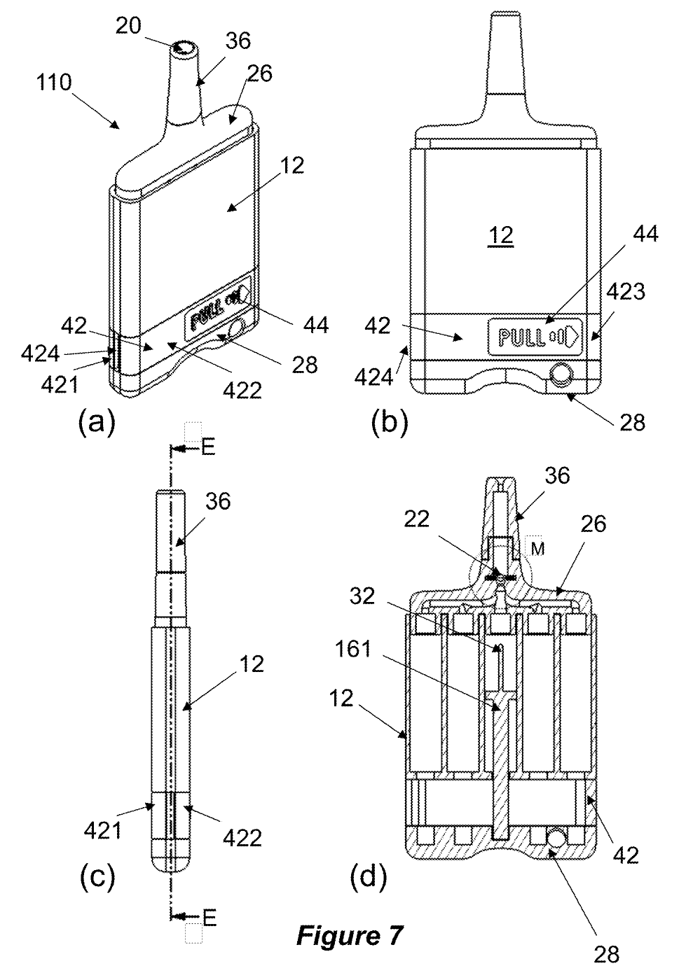

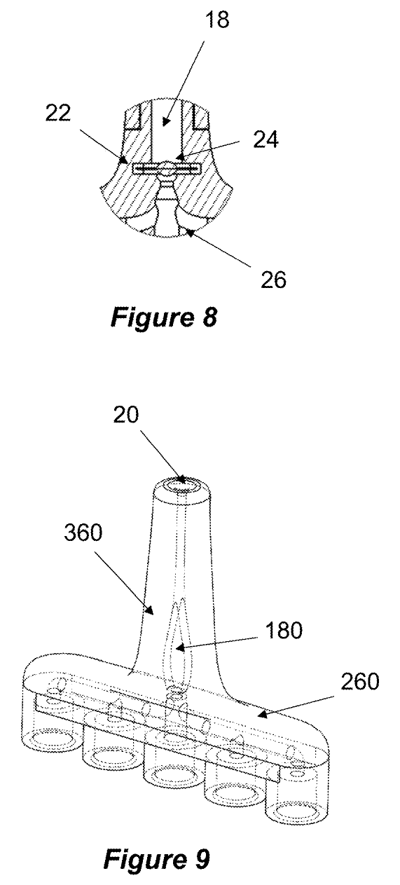

Referring now to FIGS. 7 and 8, a further embodiment of a device 110 according to the present disclosure is shown. The device 110 is again similar to the device 10 described above with reference to FIGS. 1 to 4 and like reference numerals are intended to designate like features (certain plungers 16 are not shown in FIGS. 6(d) and are omitted only to assist in an understanding of the present example embodiment). The device 110 differs from the device 10 of the earlier embodiment, however, through the inclusion of an actuation prevention mechanism and a different type of releasable seal.

In more detail, the device 10 includes an actuation prevention mechanism that inhibits movement of the plurality of plungers 16 from the first position to the second position prior to a release of the actuation prevention mechanism. In the present example embodiment, the actuation prevention mechanism comprises a blocking structure 42 releasably positioned between the handle 28 and the housing 12 which inhibits relative movement between the handle 28 and the housing 12 prior to release of the blocking structure 42. The blocking structure 42 in this embodiment is arranged to clip in place between the handle 28 and the housing 12 and includes opposing wings 421, 422 that are connected together at one lateral edge 423 of the blocking structure 42 and that are split apart at an opposite edge 424 of the blocking structure 42. The opposing wings 421, 423 are configured to locate on opposite sides, respectively, of proximal end regions of the plurality of plungers 16. The blocking structure 42 includes a surface making 44 which may indicate an instruction and/or direction for release of the blocking structure 42, such as by use of an arrow and/or use of the word “PULL”. Additionally or alternatively, the blocking structure may include a surface marking 44 that is raised or recessed to provide a gripping feature for finger engagement to release the blocking structure 42.

The actuation prevention mechanism 42 may prevent accidental discharge of the drug prior to its release from the device 110, which, by example, may be critical for persons relying on using the device for emergency assistance by administration of the drug.

The releasable seal 220 of the device 110 in this example embodiment is shown in greater detail in FIG. 8 and comprises a pierceable film adjacent the inlet 24 of the second chamber 18. In this embodiment, the actuator 32, which again comprises a projection extending from a distal end of a first plunger 161, actuates release of the releasable seal 22 by piercing the film when extending through a channel of the manifold 260 in a similar manner as described above with reference to the previous embodiments.

In preceding embodiments, the second chamber 18 is generally cylindrical in shape. In an alternative embodiment, and with reference to FIG. 9, which shows a nozzle 360 and manifold 260 for a device according to an embodiment of the present disclosure, the second chamber 180 may have a non-cylindrical shape. In this particular embodiment, the second chamber 180 has an elliptical or tear-drop prism shape. This shape of the second chamber 180 may be more suitable for accommodating drug in a powdered form and where the device is “credit card” shaped or sized as described above, in comparison to a cylindrical shape..

In some alternative embodiments, a device for administering a drug may be substantially similar to one or more of the devices according to preceding embodiments but, instead of the device having means such as plungers to pressurise fluid in the plurality of first chambers during use, the device may rather comprise a plurality of pre-pressurised first chambers. The pre-pressurised first chambers may be pre-pressurised at a predetermined pressure to allow for discharge of the drug for administration at a velocity and volume that is appropriate for a particular person or persons. The pre-pressurised first chambers may be provided by respective pressurised gas canisters, for example, and the device may include a user-actuatable trigger to cause discharge of the pressurised gas,. e.g. via a manifold of a type as described above, into a second chamber comprising drug in powdered form as described above. The pre-pressurised first chambers may be distributed within a housing of the device in a similar manner to the first chambers 14 described with respect to preceding embodiments and therefore allow the device to have a similar shape or size.

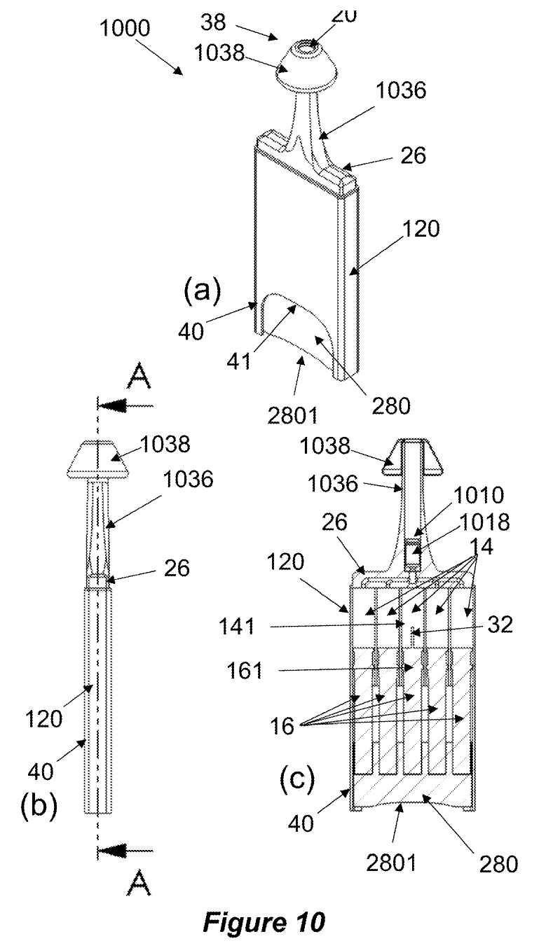

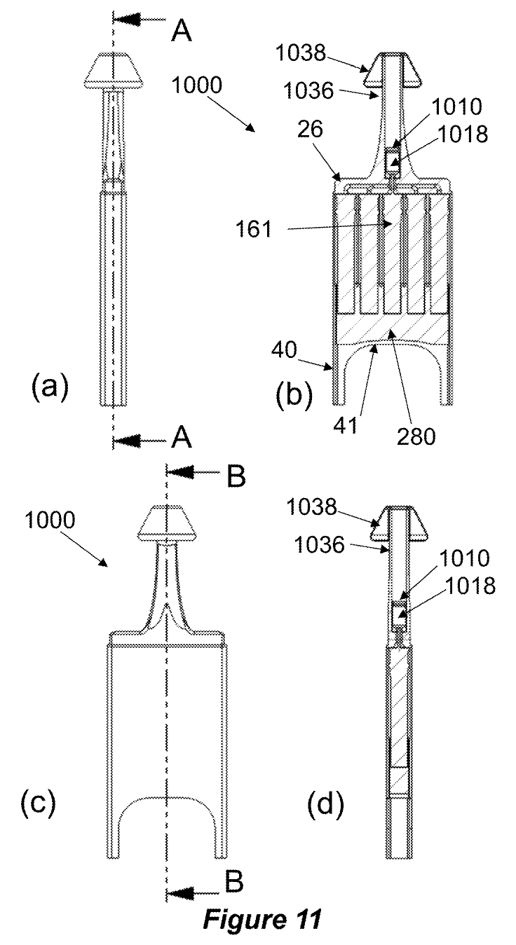

Referring now to FIGS. 10 to 13, a device 1000 according to another embodiment of the present disclosure is illustrated. The device 1000 is similar to the device 100 described with reference to FIGS. 5 and 6, and like reference numerals are intended to designate like features. The device 1000 of this embodiment differs from the device 100 of the previous embodiment in relation to the configuration of its second chamber 1018 and nozzle 1036.

In this embodiment, the device 1000 further comprises a cartridge 1010 defining the second chamber 1018 and configured to contain the drug. The cartridge 1010 may be formed separately from the housing 120 and subsequently installed in the housing 120 in selective fluid flow communication with the plurality of first chambers 14 via the releasable seal 1022.

This arrangement may reduce manufacturing costs, as the cartridge 1010 can be fabricated and sealed with the drug inside in a clean room facility, while the rest of the device 1000 can be manufactured separately with less stringent requirements, as it is not in direct contact with the drug. The sealed cartridge 1010 can then be stored and shipped separately, if required, reducing the storage space required to maintain the drug at suitable storage conditions (e.g., temperature). Preparing the sealed cartridge containing the drug reduces the likelihood of leakage, contamination and contamination of the drug by maintaining it in a sealed environment until administration.

In this embodiment, the cartridge 1010 comprises the releasable seal 1022, which releasably seals the inlet 1024 of the second chamber 1018 (the inlet 1024 being defined by the cartridge 1010).

The cartridge 1010 also defines the outlet 1020 of the second chamber 1018 and comprises a second releasable seal 1021 covering the outlet 1020. In this embodiment, the nozzle outlet 20 at the discharge end 38 of the nozzle is further downstream from the cartridge outlet 1020.

When the plungers 16 are moved from the first position to the second position, this actuates release of the second releasable seal 1021 (as well as the releasable seal 1022 at the inlet 1024) to release the fluid under pressure from the plurality of first chambers 14 into the second chamber 1018, and out through the nozzle outlet 20, thereby entraining the drug in the fluid and causing the drug to be discharged from the nozzle for administration.

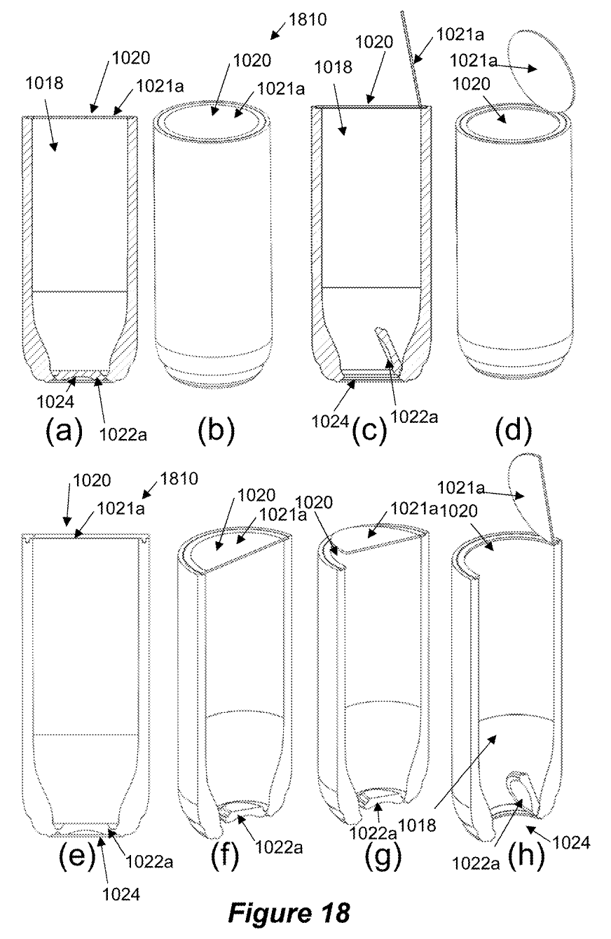

Various optional cartridges are described below, for illustrative purposes only, in relation to FIGS. 18 to 20. However, it will be understood that any suitable cartridge with releasable seals may be used.

The cartridge 1010 may be located partially or entirely in the nozzle 1036, or upstream of the nozzle 1036, between the first chambers 14 and the nozzle outlet 20.

The cartridge outlet 1020 is located at or upstream of the nozzle outlet 20 at the discharge end 38 of the nozzle 1036 that is configured to be received in a mouth and/or nose of a person.

The device 1000 is configured such that moving the plurality of plungers 16 from the first position to the second position actuates release of the second releasable seal 1021 to allow the drug to be discharged from the outlet 1020 (and nozzle 1036) for administration.

In some embodiments, the device 1000 is configured such that, upon actuation of the plungers 16, the second seal 1021 is released at the same time or after the first seal 1022. In some embodiments, the device may be configured such that the second seal 1021 is released prior to the first seal 1022.

The device 1000 may further comprises a guide cone 1038 at or near the outlet 1020, to facilitate positioning of the device 1000 to facilitate positioning of the device 1000 in the nose of a person for administration of the drug. For example, the guide 1038 may surround the nozzle outlet 20. The guide cone may be configured to provide visual and tactile guidance for correct insertion depth in the nose with instructions to insert until the guide cone seals against the nostril and/or is entirely inserted into the nostril.

The guide cone 1038 shown is substantially conical, but may alternatively define other non-conical tapered geometries.

The guide cone 1038 may be positioned around the discharge end 38 of the nozzle 1036 to facilitate positioning of the nozzle 1036 in the nose of a user.

The guide cone 1038 may be formed at least partly of resiliently flexible material. Any suitable material may be used including polymers, elastomers, silicone, or rubber, for example.

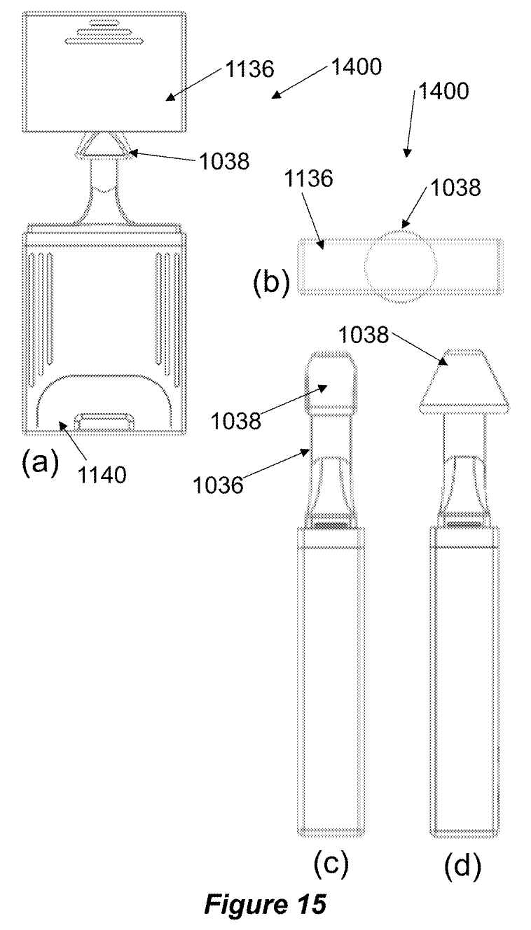

The guide cone 1038 may be at least partially formed of a resiliently deformable material and configured to partially collapse to fit within a removably couplable cap having a relatively smaller dimension in one direction relative to a width of the guide cone. That is, at least a portion of the guide cone 1038 that is formed of resiliently flexible material may be configured to be compressed to fit into the cap, as shown in FIG. 15, for example. In other words, the guide cone 1038 may have a diameter when in an uncompressed state that is greater than at least one outer dimension of the cap.

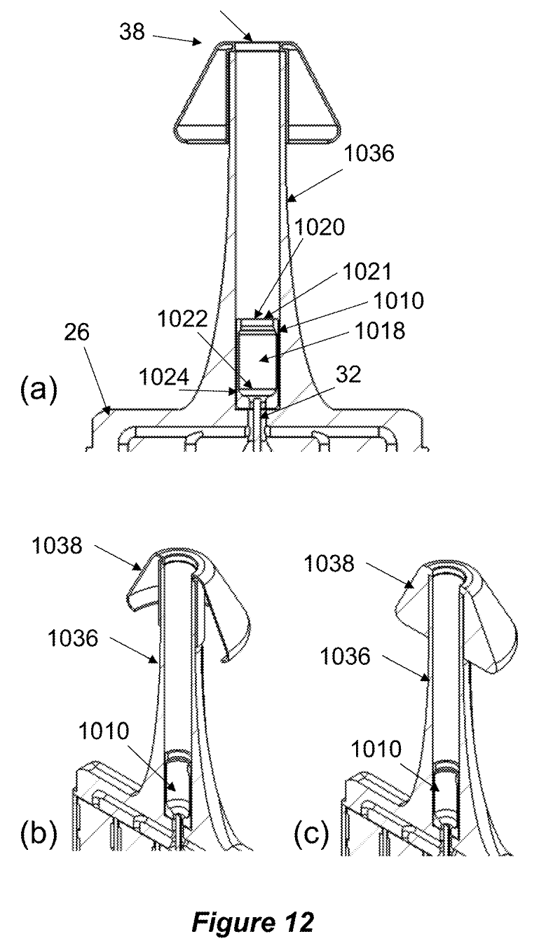

The guide cone 1038 may be substantially solid in form, as shown in FIG. 12(c), or may comprise a thin walled or shell type construction, as shown in FIG. 12(b). The guide cone 1038 may be formed together with the nozzle 1036, or alternatively, may be formed separately and attached by any suitable means, including welding, adhesive bonding, interference fit or friction fit, or being fitted over a complementary mating surface with protrusions or ridges to resist removal of the guide cone 1038, for example.

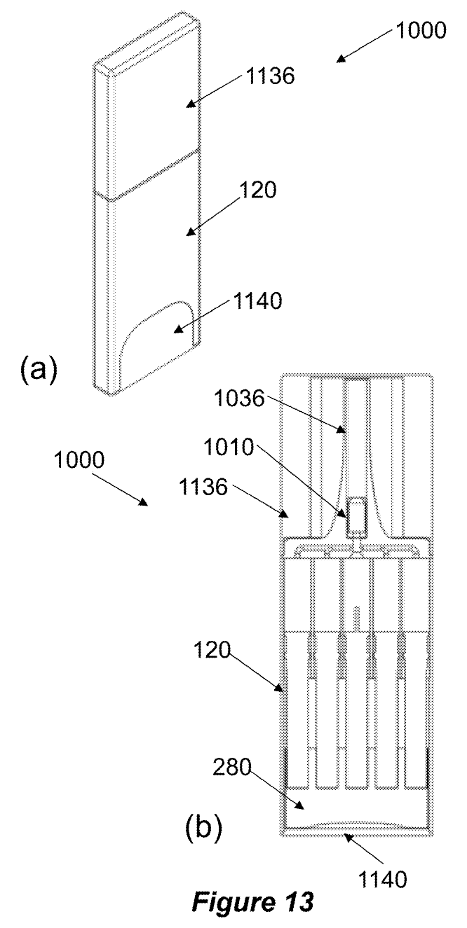

Referring to FIG. 13 (guide cone not shown), the device 1000 may further comprise one or more protective caps to protect the device 1000 and reduce the likelihood of inadvertent actuation prior to use. Two caps are shown, including a nozzle cap 1136 and a handle cap 1140.

The nozzle cap 1136 is removably couplable to the housing 120 to cover and protect the nozzle 1036. The handle cap 1140 is removably couplable to the housing to cover the handle 280 and prevent inadvertent pressing of the handle 280.

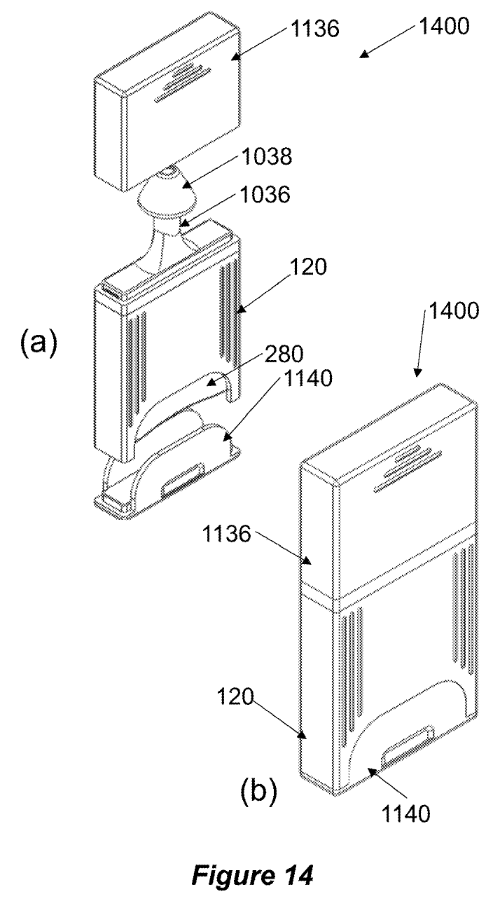

Referring to FIGS. 14 to 17, a device 1400 according to another embodiment of the present disclosure is illustrated. The device 1400 is similar to the device 1000 described with reference to FIGS. 10 to 13, and like reference numerals are intended to designate like features. The device 1400 of this embodiment differs from the device 1000 of the previous embodiment in relation to its proportions and in the configuration of its nozzle 1436.

FIG. 14 illustrates the nozzle cap 1136 and handle cap 1140, similar to the caps of device 1000. The nozzle cap 1136 and handle cap 1140 may be coupled to the housing 120 by any suitable means, including a snap fit connection, catch, or interference fit, for example. In some embodiments, the nozzle cap 1136 and handle cap 1140 may be connected to the housing 120 with frangible joints which can be broken to allow use of the device 1400.

FIG. 15 illustrates the diameter of the guide cone 1038 in an uncompressed state being wider than the thickness of the device 1400 and nozzle cap 1136 (FIGS. 15(b) and 15(d)), and then being compressed as shown in FIG. 15(c) to fit into the nozzle cap 1136 as shown in FIG. 15(a).

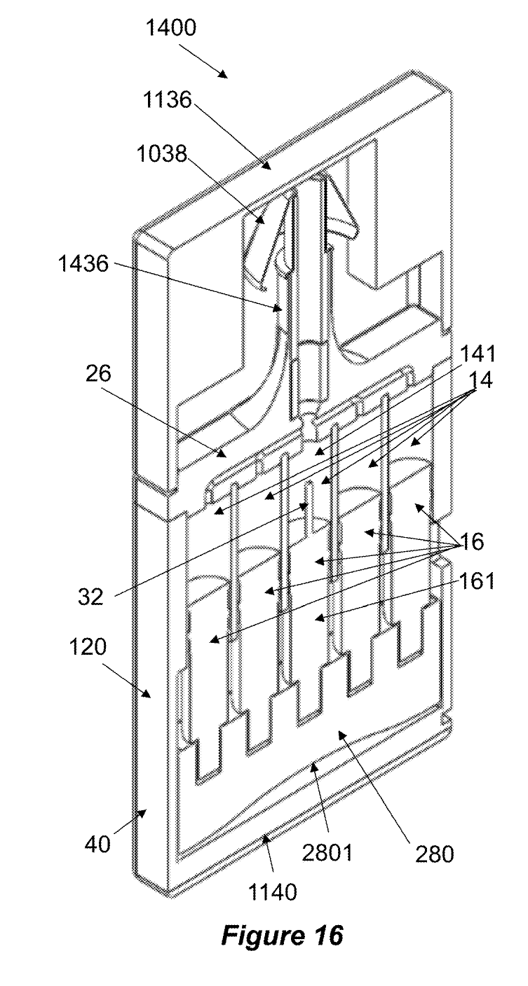

FIG. 16 shows a cross section of the device 1400 in isometric perspective view illustrating the device 1400 prior to removal of the caps 1136, 1140 and actuation of the device.

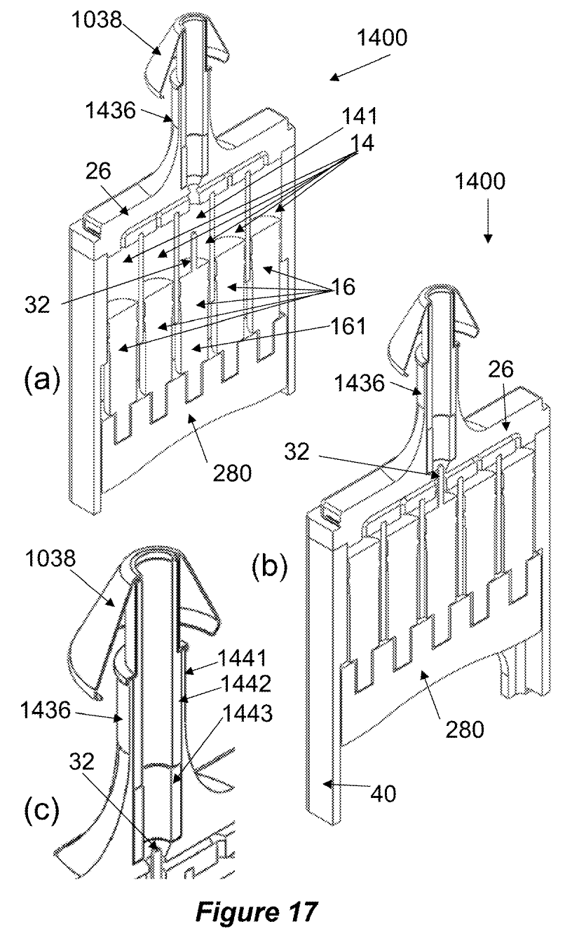

The nozzle 1436 is formed of multiple components, in contrast with the single piece nozzle shown in previous embodiments. In some embodiments, the nozzle may be integrally formed with the manifold and housing. In some embodiments, the nozzle may be formed of separate components assembled together to form the nozzle, as shown in FIGS. 16 and 17,

For example, the nozzle 1436 comprises a first component 1441 integrally formed with the manifold and defining an internal bore to receive the other components. The nozzle 1436 further comprises a second component 1442 and a third component 1443 formed separately and assembled within the bore of the first component 1441 to form the nozzle and define a seat for a cartridge 1010 and a passage to allow fluid flow through the nozzle 1436.

In some embodiments, it may be preferable to form the nozzle in one part to simplify fabrication. In some embodiments, a multicomponent nozzle assembly may be preferable, as it allows for the use of different materials with different properties which may be more suitable (e.g., dimensional stability), and allows for separate machining, which may allow for more precise tolerances, if required. The components may be formed of any suitable materials including polymers, ceramics, or metals, such as stainless steel, for example.

The components 1441, 1442, 1443 may be fitted together with adhesive and/or an interference fit or friction fit, for example.

Similarly, the cartridge 1010 may be installed with adhesive and/or an interference fit or friction fit, for example.

In some embodiments, the nozzle may be formed separately to the manifold and housing, and connected to the manifold to form the device. In some embodiments, the cartridge may comprise the nozzle. In other words, the cartridge may define the nozzle, or the nozzle may define the cartridge. This allows the cartridge and nozzle to be fabricated separately and sealed with the drug contained within the cartridge, and then fitted to the manifold/housing, which in turn allows the nozzle and cartridge to be replaced with a new nozzle/cartridge for reuse of the housing/plunger assembly.

Referring now to FIGS. 18 to 20, various cartridges are shown according to some embodiments. Any other suitable cartridges with various releasable seals may be used with the device.

FIG. 18 illustrates a cartridge 1810 with a membrane seal 1022a at the inlet 1024 and a membrane seal 1021a at the outlet 1020. The membranes may be released or burst by an actuator (e.g., push rod 32), or alternatively set to release when a predetermined pressure differential across the membrane is reached.

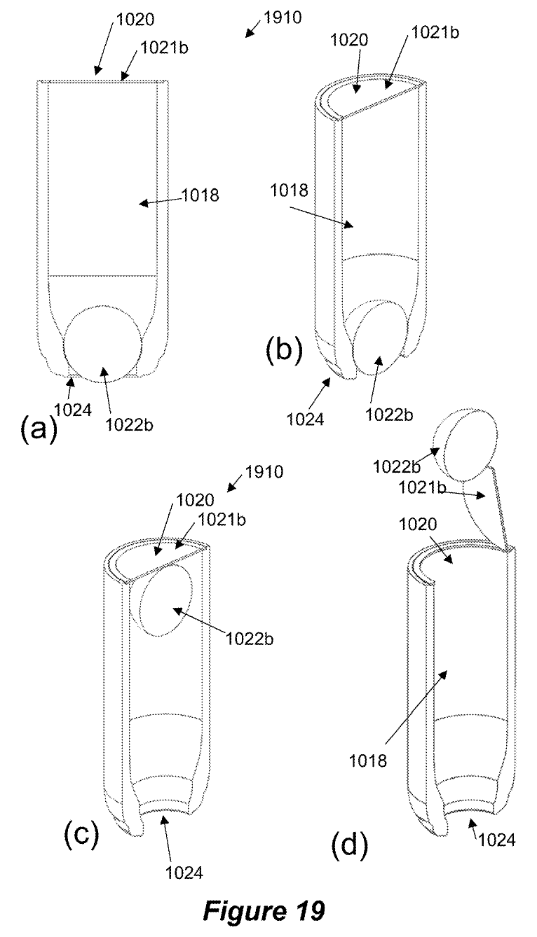

FIG. 19 illustrates a cartridge 1910 with a membrane seal 1021b at the outlet 1020, and a ball seal 1022b at the inlet 1024. The ball seal 1022b comprises a spherical element seated in a groove at the inlet 1024 with an interference fit which provides sufficient resistance to maintain the seal under pressure until dislodged by the actuator. The membrane seal 1021b may be released or burst by an actuator (e.g., push rod 32), or alternatively set to release when a predetermined pressure differential across the membrane is reached.

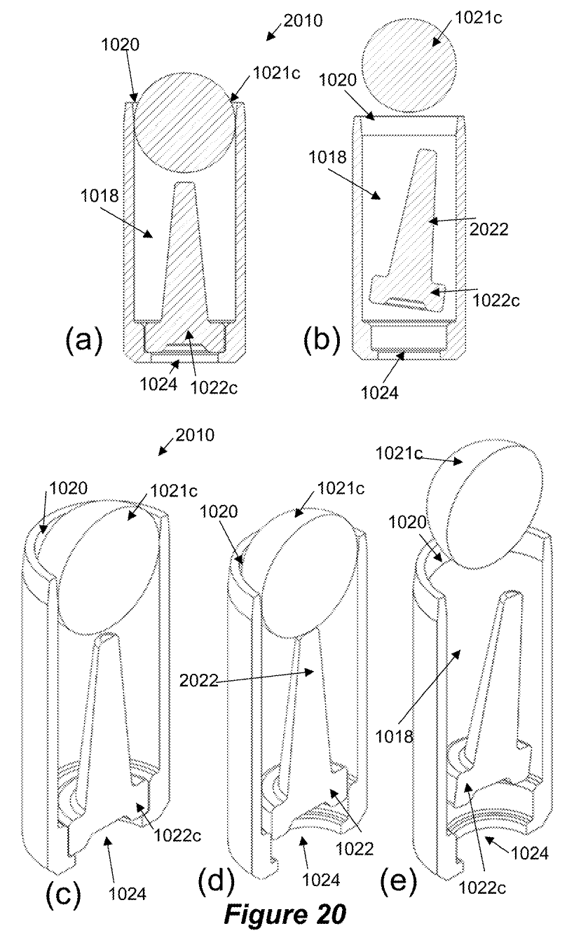

FIG. 20 illustrates a cartridge 2010 with a ball seal 1021c at the outlet 1020, and a slide pin seal 1022c at the inlet 1024. The slide pin seal 1022c comprises a cylindrical portion seated against a corresponding cylindrical bore at the inlet 1024. The slide pin seal 1022c is seated in the bore with an interference fit or friction fit with sufficient friction to resist the increasing pressure in the first chambers 14 until dislodged by the actuator sliding it out of the bore and into the second chamber 1018.

The ball seal 1021c comprises a spherical element seated in a groove at the outlet 1020 with an interference fit which provides sufficient resistance to maintain the seal under pressure until dislodged by a slide pin 2022 extending from the slide pin seal 1022.

The length of the slide pin 2022 may be chosen to contact and dislodge the ball seal 1021c at the same time as the slide pin seal 1022c is released, or after the slide pin seal 1022c is released, or before the slide pin seal 1022c is released, so as to control the release of the fluid into the second chamber 1018, out through the outlet 1020 and out through the nozzle 1036 for administration of the drug.

Any of the seals described (or any other suitable seal) may be used to replace any one of the seals in any other described embodiment. For example, the slide pin seal may be used to break a membrane seal at the outlet.

It will be appreciated that many other seal types may be used to seal the cartridge and be released by any suitable release mechanism.

Any of the features described in relation to any one of the embodiments may be used in combination with or instead of features of other described embodiments.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A device for nasally administering a drug to a user, the device comprising:
a housing;
a plurality of first chambers located in the housing, each of the first chambers containing a fluid and arranged in a parallel configuration along a width direction of the housing;
a plurality of plungers, each plunger of the plurality of plungers movable within a respective first chamber of the plurality of first chambers from a first position to a second position to increase a pressure of the fluid contained in the first chamber;
a second chamber configured to contain the drug and having an inlet and an outlet, wherein at least part of the second chamber is located in a nozzle of the device, the nozzle configured to be received at least partially in the nose of the user;
at least one releasable seal located at or adjacent the inlet of the second chamber between the plurality of first chambers and the drug contained in the second chamber, the second chamber, including the outlet, being in selective fluid flow communication with the plurality of first chambers via the releasable seal; and
an actuator comprising a projection extending from a distal end of a first plunger of the plurality of plungers and configured to actuate release of the releasable seal,
wherein moving the plurality of plungers from the first position to the second position causes the actuator to actuate release of the releasable seal to release the fluid under pressure from the plurality of first chambers into the second chamber, thereby entraining the drug in the fluid and causing the drug to be discharged from the outlet for administration;
wherein the plurality of first chambers consists of an odd number of first chambers including a central first chamber and a plurality of further first chambers distributed evenly on opposite sides of the central first chamber, wherein the first plunger of the plurality of plungers is a central first plunger moveable within the central first chamber;
wherein the device comprises a manifold that fluidly connects the plurality of first chambers to the inlet of the second chamber, wherein the projection extending from the distal end of the central first plunger is configured to extend through the manifold to actuate release of the releasable seal.

2. The device of claim 1, wherein the housing is non-cylindrical and each first chamber of the plurality of first chambers is substantially cylindrical.

3. The device of claim 1, wherein the housing has a substantially rectangular cross sectional profile.

4. The device of claim 1, wherein the device has a length dimension of less than 86mm and a width dimension of less than 55mm when the plurality of plungers are in the first position.

5. The device of claim 1, comprising a common handle connected to each of the plurality of plungers, wherein the plurality of plungers are movable by the handle from the first position to the second position.

6. The device of claim 5, comprising a cover portion for receiving the handle when the plurality of plungers are in the second position, wherein the cover portion substantially conceals the handle when the plurality of plungers are in the second position.

7. The device of claim 6, wherein the cover portion is configured to guide the handle to move the plurality of plungers substantially uniformly from the first position to the second position.

8. The device of claim 5, wherein the handle is accessible only proximate the central first plunger when the plurality of plungers are in the first position.

9. The device of claim 5, wherein the handle comprises a rest proximate the central first plunger, wherein the rest comprises a recess in a surface of the common handle.

10. The device of claim 1, wherein the actuator of the first plunger is configured to actuate release of the releasable seal only when the first plunger has moved at least 50%, at least 60%, at least 70%, at least 80% or at least 90% of a distance between the first position and the second position.

11. The device of claim 1, wherein the releasable seal comprises a pierceable film, the actuator actuating release of the releasable seal by piercing the film.

12. The device of claim 1, wherein the releasable seal comprises an interference fit ball, the actuator actuating release of the releasable seal by dislodging the ball from the interference fit.

13. The device of claim 1, wherein the drug is a nasally delivered drug powder and wherein the fluid contained in each of the first chambers is a gas.

14. The device of claim 1, further comprising a cartridge defining the second chamber and containing the drug, wherein the cartridge is formed separately from the housing and subsequently installed in the housing in selective fluid flow communication with the plurality of first chambers via the releasable seal between the plurality of first chambers and the second chamber.

15. The device of claim 1, wherein the releasable seal between the plurality of first chambers and the second chamber is a first releasable seal and wherein the device comprises a second releasable seal covering the outlet of the second chamber, wherein moving the plurality of plungers from the first position to the second position actuates release of the second releasable seal to allow the drug to be discharged from the outlet for administration, and wherein the device is configured such that the second seal is released at the same time or after the release of the first seal.

16. The device of claim 1, wherein the outlet of the second chamber is located at, or upstream of, a discharge end of the nozzle.

17. The device of claim 1, wherein a guide cone is positioned around a discharge end of the nozzle to facilitate positioning of the nozzle in the nose of the user, wherein the guide cone is formed at least partly of resiliently flexible material, wherein the device comprises a cap configured to cover the nozzle, wherein at least a portion of the guide cone that is formed of resiliently flexible material is configured to be compressed to fit into the cap, and wherein the guide cone has a diameter when in an uncompressed state that is greater than at least one outer dimension of the cap.

18. The device of claim 1, wherein the odd number of first chambers is 3, 5 or 7 first chambers.

19. A device for nasally administering a drug to a user, the device comprising:
a housing;
a plurality of first chambers located in the housing, each of the first chambers containing a fluid and arranged in a parallel configuration along a width direction of the housing;
a plurality of plungers, each plunger of the plurality of plungers movable within a respective first chamber of the plurality of first chambers from a first position to a second position to increase a pressure of the fluid contained in the first chamber;
a second chamber configured to contain the drug and having an inlet and an outlet, wherein at least part of the second chamber is located in a nozzle of the device, the nozzle configured to be received at least partially in the nose of the user;
at least one releasable seal located at or adjacent the inlet of the second chamber between the plurality of first chambers and the drug contained in the second chamber, the second chamber, including the outlet, being in selective fluid flow communication with the plurality of first chambers via the releasable seal; and
an actuator comprising a projection extending from a distal end of a first plunger of the plurality of plungers and configured to actuate release of the releasable seal, wherein the releasable seal comprises an interference fit ball, the actuator actuating release of the releasable seal by dislodging the ball from the interference fit;
wherein moving the plurality of plungers from the first position to the second position causes the actuator to actuate release of the releasable seal to release the fluid under pressure from the plurality of first chambers into the second chamber, thereby entraining the drug in the fluid and causing the drug to be discharged from the outlet for administration;
wherein the device comprises a manifold that fluidly connects the plurality of first chambers to the inlet of the second chamber, wherein the projection extending from the distal end of the first plunger is configured to extend through the manifold to actuate release of the releasable seal.

20. A device for nasally administering a drug to a user, the device comprising:
a housing;
a plurality of first chambers located in the housing, each of the first chambers containing a fluid;
a plurality of plungers, each plunger of the plurality of plungers movable within a respective first chamber of the plurality of first chambers from a first position to a second position to increase a pressure of the fluid contained in the first chamber;
a second chamber configured to contain the drug and having an inlet and an outlet, wherein at least part of the second chamber is located in a nozzle of the device, the nozzle configured to be received at least partially in the nose of the user;
at least one releasable seal located at or adjacent the inlet of the second chamber between the plurality of first chambers and the drug contained in the second chamber, the second chamber, including the outlet, being in selective fluid flow communication with the plurality of first chambers via the releasable seal;
an actuator comprising a projection extending from a distal end of a first plunger of the plurality of plungers and configured to actuate release of the releasable seal; and
a common handle connected to each of the plurality of plungers, wherein the plurality of plungers are movable by the common handle from the first position to the second position;
wherein moving the plurality of plungers from the first position to the second position causes the actuator to actuate release of the releasable seal to release the fluid under pressure from the plurality of first chambers into the second chamber, thereby entraining the drug in the fluid and causing the drug to be discharged from the outlet for administration; and
wherein the device has a length dimension of less than 86mm and a width dimension of less than 55mm when the plurality of plungers are in the first position.
